(19) United States
(12) Patent Application Publication
(10) Pub. No.: US 2022/0105512 A1
(43) Pub. Date: Apr. 7, 2022

(54) METHOD AND DEVICE FOR THE QUALITATIVE AND/OR QUANTITATIVE MEASUREMENT OF ODORS PRODUCED BY A PHYSICOCHEMICAL TRANSFORMATION OF A PRODUCT

(71) Applicant: ARYBALLE, Grenoble (FR)

(72) Inventor: Cyril HERRIER, GRENOBLE CEDEX 09 (FR)

(21) Appl. No.: 17/421,103

(22) PCT Filed: Jan. 7, 2020
(86) PCT No.: PCT/FR2020/050020

§ 371 Date: Jul. 7, 2021

(30) Foreign Application Priority Data
Jan. 8, 2019 (FR) 19 00153

(51) Int. Cl. B01L 3/00; G01N 21/552; G01N 33/00

(52) U.S. Cl. CPC B01L 3/502715; G01N 21/553; G01N 33/0031; G01N 33/0006; G01N 33/0029; B01L 3/50273; B01L 2400/06; B01L 2200/026; B01L 2200/0689; B01L 2200/16; B01L 2300/0663; B01L 2400/049; B01L 3/502738

(57) ABSTRACT

A method for the qualitative and/or quantitative measurement of odors present in a chamber for transformation (cooking or fermentation) of a product makes it possible to prevent the measurement environment from contamination by measurement-interfering elements produced by the transformation and released in the chamber for transformation. The method makes use of a chamber for transformation, for example an oven, and of an electronic nose, pipes a fluidic system, and a removal chamber for removing the measurement-interfering elements. The method includes a measurement mode M and a cleaning mode N. The cleaning mode comprises the following steps: -N0- optionally, flushing the chamber for transformation, preferably by ventilating it; -N1- flushing the measurement chamber with a fluid, preferably ambient air; -N2- flushing the removal chamber with a fluid, preferably ambient air; and -N3- optionally, transferring at least a portion of the flushing fluid from the removal chamber into the chamber for transformation.

### TECHNICAL FIELD

The field of the invention is that of using electronic noses capable of recognizing, differentiating, and detecting odors, scents, odorous substances, odorous molecules.

It more specifically concerns the measurement, with these electronic noses, of the odors emitted inside a chamber for physicochemical transformation of a product under the effect of certain environmental conditions such as, for example, pressure, temperature, humidity. This physicochemical transformation may in particular be cooking or fermentation. The cooking chamber may be for example an oven, and the fermentation chamber a fermentation tank. The product concerned is any product, in particular a food product. An odor measurement, as long as it is reliable, precise, and reproducible, aims to allow monitoring the transformation (e.g. cooking, fermentation) of a product, for example food, in a transformation/cooking-fermentation chamber.

The invention thus relates to a device for the qualitative and/or quantitative measurement of odors produced during a transformation (e.g. cooking, fermentation).

### PRIOR ART

The electronic noses used in the method and device according to the invention can be of different types.

For example, these electronic noses can be bioelectronic noses comprising non-specific biosensors, which operate according to a principle of surface plasmon resonance (SPR) imaging. As a non-limiting illustration of these bioelectronic noses, reference is made here to the device designed, manufactured, and marketed under the name NeOse® by the company Aryballe.

The NeOse® is an opto-electro-mechanical system comprising:
a suction subsystem comprising a mini-pump which captures a sample volume of gas. It is composed of a mechanism controlled to ensure the fluidic function. Its upstream length is minimized to avoid contamination or transformation of the sample as much as possible. The flow rate is adjustable. Typical values are around 60 ml/minute.
a sensory subsystem comprising a reactive stage in which specific “adsorbent” molecules are deposited and attached (covalent bonds) in pads on a layer of gold. This reactive stage constitutes a measurement chamber comprising a support on which the pads of adsorbent molecules, or receptors, form the same number of sensitive areas. These sensitive areas offer a variety of temporary inter-molecular bonds between the molecules present in the expansion space of the measurement chamber and the adsorbing molecules of the receptors.
a conventional reflection-based SPR stage, composed of a diode-type emitter of a monochromatic incident beam of which the emission is polarizable, a glass prism on which the reactive stage is deposited, and a CCD camera [Charge-Coupled Device, or in French “dispositive à transfert de charge” (DTC)]. The beam passes through the prism, impinges on the dioptric glass-gold interface, and is reflected back to the CCD camera arranged in the symmetrical direction.
an application processor stage (motherboard), composed of a microprocessor capable of booting a micro-linux operating system (OS). The CCD camera is installed via a USB port on the motherboard.
an Input/Output (I/O) communication stage: Block/Transfer (B/T), Wifi, wired.

These sensitive areas, or functional receptors, modify the local refractive properties according to their percentage of occupancy by the odorous molecules adsorbed. This variation in the refractive index explains the variations in intensity (gray levels, e.g. from 0 to 255) obtained in the control of the CCD camera.

In addition to the electronic nose, for example of the NeOse® bioelectronic type, the measurement according to the invention of cooking odors involves a cooking chamber which can be an oven or any other cooking appliance, pipes providing fluid communication between the electronic nose and the oven, and a fluidic system managing this communication.

It turns out that a cooking chamber, in particular for food products, is a humid and soiled environment. The electronic nose makes its measurement on gas samples from this chamber. However, the humidity, particles, and soiling produced are elements that interfere with the measurement from this electronic nose. In particular, when it concerns a bioelectronic nose based on SPR technology, the water, particles, and soiling are liable to damage the prism and sensors in particular of the bioelectronic nose.

### Technical Problem—Aims of the Invention

In this context, the problem therefore arises of countering the harmful influence, on the measurement, of these interfering elements which may be humidity, particles, and soiling of the chamber for transformation (e.g. cooking, fermentation) of a product.

In addition, it is important that the remedy provided against interference with the measurement by the aforementioned elements is also effective against interfering elements composed of certain chemical species produced by transformation of the product (e.g. cooking, fermentation), which are particularly adhesive and thus tend to attach themselves inside the fluid communicating pipes. In the case of food products, this is typically fat.

In addition, this remedy must not only be effective, but also must be easy to implement and economical.

The invention is thus intended to satisfy at least one of the aims set forth below.

One of the essential aims of the invention is to provide a method for the qualitative and/or quantitative measurement of odors present in a chamber for transformation (e.g. cooking, fermentation) of a product, this method making it possible to avoid contamination of the measurement medium by elements produced by the transformation (e.g. cooking, fermentation) and released in the chamber for transformation (e.g. cooking, fermentation).

One of the essential aims of the invention is to provide a method for the qualitative and/or quantitative measurement of odors present in a chamber for transformation (e.g. cooking, fermentation) of a product, which makes it possible to avoid, in a practical, simple, efficient, and economical manner, contamination of the measurement medium by elements contained in the chamber for transformation (e.g. cooking, fermentation).

Another essential aim of the invention is to provide a method for the qualitative and/or quantitative measurement of odors present in a chamber for transformation (e.g. cooking, fermentation) of a product, which is simple, economical, and efficient with regard to the interfering elements produced by the transformation (e.g. cooking, fermentation), such as water and the adhering fats which clog the passage of the communicating pipes between the chamber and the electronic nose.

One of the essential aims of the invention is to provide a method for the qualitative and/or quantitative measurement of odors present in a chamber for transformation (e.g. cooking, fermentation) of a product, making it possible to control the transformation (e.g. cooking), for example of a food product, by taking into account the measured odors and their changes during cooking.

One of the essential aims of the invention is to provide an efficient, robust, simple, and economical device for the qualitative and/or quantitative measurement of odors present in a chamber for transformation (e.g. cooking, fermentation) of a product, in particular for implementing the method as referred to in the above paragraphs.

### Description of the Invention

These aims and others are achieved by the invention, which firstly relates to a method for the qualitative and/or quantitative measurement of odors present in a chamber for transformation, preferably cooking or fermentation, of a product, wherein the following are used:
at least one chamber for transformation;
at least one electronic nose provided with at least one measurement chamber comprising at least one sensor, capable of supplying a signal representative of the development of odors during this transformation;
pipes and a fluidic system making it possible to place the measurement chamber in communication with the chamber for transformation, so that samples of the atmosphere of the latter chamber can be transferred into the measurement chamber, where these samples are then brought into contact with the sensor or sensors;
at least one removal chamber connected to the communicating pipes, interposed between the chamber for transformation and the electronic nose and capable of eliminating all or part of any measurement-interfering elements present in the chamber for transformation and therefore also in the samples transferred to the measurement chamber;
said method comprising at least one measurement mode M and at least one cleaning mode N comprising the following steps:
→Mode M:
M.I/ optional pre-measurement phase comprising the following steps:
-m1- at least one sample is transferred from the chamber for transformation (e.g. cooking or fermentation) into the measurement chamber via the removal chamber;
-m2- at least one odor measurement is carried out on the sample;
-m3- the sample is discharged from the measurement chamber;
steps m1,m2, and m3 preferably taking place continuously;
M.II/ the product is transformed (e.g. cooking or fermentation) in the chamber for transformation;
M.III/ measurement phase comprising steps m1, m2, and m3;
steps m1, m2 and m3 preferably taking place continuously;
M.(III)ₓ/ optional repetition, with x being an integer >1;
M.IV/ optionally, the transformation (e.g. cooking or fermentation) is controlled according to a transformation temperature adjustment setpoint and/or a transformation stopping setpoint, and optionally according to a transformation duration setpoint, taking into account the odor measurement(s) in M.III;
M.V/ the transformation (e.g. cooking or fermentation) is stopped;
→Mode N:
-N0- optionally, flushing the interior of the chamber for transformation (e.g. oven or fermentation tank), preferably by ventilating it;
-N1- flushing the measurement chamber with a fluid, preferably ambient air;
-N2- flushing the removal chamber with a fluid, preferably ambient air;
-N3- optionally, transferring at least a portion of the flushing fluid from the removal chamber into the chamber for transformation;
→optionally, an additional mode N is implemented before mode M.

It is to the credit of the inventors to have designed and implemented an interesting application of an electronic nose, e.g. a bioelectronic nose, for monitoring odors that develop during the transformation (e.g. cooking or fermentation) of a product, for example a food product, in a chamber for transformation (e.g. cooking or fermentation) such as an oven or a fermentation tank. The measurement method of the invention offers the advantage of avoiding the interference caused by certain elements present in the chamber for transformation (e.g. cooking or fermentation), such as the humidity and fats typical of a food product. This makes it possible to increase the sensitivity, precision, and reliability of the measurement by the electronic nose.

In addition, the invention is noteworthy in that it makes it possible to eliminate, or at the very least to considerably reduce, the fouling by interfering elements (e.g. fat/water), of the removal chamber, the communicating pipes, and the associated fluidic system. The invention provides a simple, efficient, and inexpensive procedure for cleaning the pipes, the removal chamber, and the fluidic system.

It is interesting to note that this cleaning procedure can easily be implemented not only between two transformations (e.g. cooking or fermentation), but also between two measurements during the same transformation (e.g. cooking or fermentation).

Finally, the fact that the electronic nose is located outside the cooking chamber makes it possible to measure odors at room temperature. In addition, this protects the sensors of the electronic nose from the condensation phenomenon which inevitably occurs in the transformation (e.g. cooking or fermentation) of products rich in water, such as food products.

The qualitative and/or quantitative measurement of odors from a chamber for transformation (e.g. cooking or fermentation) and more precisely from the atmosphere of this chamber, can be considered comparable to a measurement of the signature of the odor of this atmosphere.

Such a measurement is preferably carried out on an atmosphere sample comprising as little as possible and ideally no interfering element such as water, dirt, soiling, or particles, which are not part of the odor of the measurement sample and which, on the contrary, run the risk of distorting the measurement.

The method according to the invention makes it possible to meet this technical requirement.

Moreover, such a measurement of the signature of an odor from the chamber for transformation (e.g. cooking or fermentation) is advantageously implemented according to a measurement mode M comprising a phase M.III-Ref/ in which:
the measurement of odors from the chamber for transformation is made, at each site n each corresponding to an electronic nose sensor, relative to a reference measurement of a stable reference atmosphere isolated from the transformation atmosphere, this reference atmosphere preferably being the outside air;
two sets of time-response signals are measured simultaneously or sequentially at contiguous periods: a first set comprising the time-response signals Sₙ^{(a)}(ti), Sₙ^{(a)}(ti) corresponding to a time-response signal measured at site n, at time ti, in the reference atmosphere, a second set comprising the time-response signals Sₙ^{(c)}(ti), Sₙ^{(c)}(ti) corresponding to a time-response signal measured at site n, at time ti, in the atmosphere of the chamber;
preferably, at each measurement time, these two sets of signals are processed for each measurement site to obtain:
a raw curve by calculating:
   the difference d1: sₙ(ti)=sₙ^{(c)}(ti)−sₙ^{(a)}(ti);
   or the difference d2: sₙ^{(c)}(ti)−<sₙ^{(a)}(ti)>pₘ, where <sₙ^{(a)}(ti)>pₘ is the mean value of the measurement for site n in air in the completed period Pm (Pm is about 1 second to a few seconds for example);
   d2 being preferred;
and, optionally, a normalized curve by calculating a norm, preferably the min-max norm, to detect the formation of an odor, with snormₙ(ti)=[sₙ(ti)−minₙ{sₙ(ti)}]/[maxₙ{sₙ(ti)}−minₙ{sₙ(ti)}]; snorm being between 0 and 1.

In accordance with this mode of implementing the odor measurement according to the invention, each sensor of the electronic nose (corresponding to a sensitive area, i.e. a receptor of the measurement chamber) successively produces during the measurement: on the one hand, a time-response signal Sₙ^{(a)(ti)}: measurement of site (or sensor) n at time ti in the reference atmosphere, preferably outside air,
and, on the other hand, a time-response signal Sₙ^{(c)(ti)}: measurement of site (or sensor) n at time ti in the transformation atmosphere. The site (or sensor) n is the measurement site comprising, on the one hand, reference outside air which is separated in a fluidtight manner from odors from the chamber for transformation, and on the other hand, the chamber for transformation.

According to the invention, the reference measurement and the measurement of odors from the cooking chamber can be carried out in two ways.

A first way consists of ensuring that the electronic nose sniffs at least one sample of reference outside air, and at least one sample of the atmosphere of the chamber for transformation.

This sniffing is preferably carried out by means of separate communicating pipes and fluidic system. For example, the electronic nose is connected to the reference outside air by communicating pipes provided with at least one valve (fluidic system) controlling the sniffing of the reference outside air or the air devoid of transformation odor to be measured. In addition, this electronic nose communicates with the cooking chamber via communicating pipes equipped with a valve (fluidic system) controlling the sniffing of the transformation atmosphere.

According to the second method, sequential sniffing of the reference outside air and of the transformation atmosphere to be measured is performed. To do this, for example, a same valve and same communicating pipes (fluidic system) capable of sequentially inhaling the reference outside air (non-interfering air) and then the cooking atmosphere are used. This requires cleaning and flushing the communicating pipes between each inhalation to avoid contamination across measurements.

According to another aspect, the invention relates to a method for controlling a transformation, preferably a cooking or a fermentation, making use of the aforementioned method for measurement and wherein:
optionally, at least one standard pattern of odors measured during the transformation corresponding to a given transformation of a given product to be transformed, is determined beforehand by calibration;
optionally, this standard pattern is kept in memory;
odor patterns are measured continuously according to mode M during transformation of a same product to be transformed;
these measured patterns are compared with the standard pattern and/or these measured patterns are compared with the previously measured patterns;
the transformation is controlled according to the result of this comparison.

According to another aspect, the invention relates to a device for implementing the method according to the invention.

The features set forth in the following paragraphs may optionally be implemented. They may be implemented independently of one another or in combination with one another.

In one particular embodiment, the electronic nose is a bioelectronic nose operating by surface plasmon resonance (SPR) imaging.

According to an advantageous embodiment of the invention, the cooked product or product to be cooked is a foodstuff which, during transformation (cooking or fermentation), generates measurement-interfering elements selected from the group comprising—ideally consisting of—the following elements: water, fat, carbonaceous substances, and mixtures thereof.

In another particular embodiment, the chamber for transformation is an oven. In such a case, measurement mode M may comprise a preliminary step of preheating the oven.

In an optimized embodiment, the removal chamber is a container into which leads at least one pipe Cf in communication with the chamber for transformation (cooking or fermentation) and at least one pipe Cn in communication with the electronic nose; the free end of pipe Cf preferably being located below the free end of pipe Cn inside the container. In the case where pipes Cf & Cn enter the container through its upper end; the free end of pipe Cf is even more preferably located in the lower half of the container, while the free end of pipe Cn is even more preferably located in the upper half of the container.

In an optimized variant of this embodiment, cleaning mode N comprises a step N4 in which:
the pipe Cn in communication with the electronic nose is replaced by a clean pipe Cn,
and/or the inside of this pipe Cn is cleaned using a cleaning fluid.

Preferably, step -m1- is carried out by means of at least one pump, preferably a pump equipping the electronic nose and capable of suctioning the sample coming from the oven.

Preferably, step N2 is carried out by means of at least one blowing pump connected, by at least one communicating pipe, to the interior of the removal chamber and capable of propelling a flushing fluid therein.

In one embodiment of step N2, the flushing fluid is not propelled to the interior of the removal chamber, the pipe connecting the blowing pump to the interior of the removal chamber then being equipped either with a 2-way valve placed in the closed position, or with a valve comprising 2 channels and at least a 3rd channel bypassing the removal chamber, for the flushing fluid propelled by the blowing pump, said 3rd bypassing channel being placed in the open position.

In another embodiment of step N2, the flushing fluid is propelled to the interior of the removal chamber, the pipe connecting the blowing pump to the interior of the removal chamber then being equipped either with a 2-way valve placed in the open position, or with a valve comprising 2 channels and at least a 3rd channel bypassing the removal chamber, for the flushing fluid propelled by the blowing pump, said 3rd bypassing channel being placed in the closed position.

### Definitions

Throughout the present description, any singular indiscriminately designates a singular or a plural.

The definitions given below by way of example can help in interpreting the present description:
“odors” means odors, scents, odorous substances, odorous molecules, and mixtures thereof.
“oven” designates any cooking appliance operating in particular by induction, microwave, Joule effect, gas combustion, steam (pressure cooker). This applies to both closed heating chambers and open chambers such as cooking appliances under a hood comprising a container containing the product to be cooked, a means for heating the container and the product to be cooked, and a hood extracting the volatile compounds from cooking.
“Electronic nose” designates any type of electronic nose operating by SPR, optical, electronic and/or mechanical technology.
“Removal chamber” designates a container constituting a trap or a filter for eliminating elements interfering with the measurement.
“sensor” designates a specific sensor as well as a non-specific sensor. In certain electronics, namely the bioelectronic nose based on SPR, the non-specific sensors are the sensitive areas which are also called functional receptors.

### BRIEF DESCRIPTION OF DRAWINGS

Other features, details and advantages of the invention will be apparent from reading the detailed description below of some exemplary implementations of the method according to the invention by means of a preferred embodiment of the device according to the invention, with reference to the accompanying drawings, in which:

FIG. 1 shows a diagram of the device according to the invention.

FIG. 2 shows an experimental diagram of implementing the method according to the invention.

FIG. 3 shows an experimental diagram of implementing the method according to the invention, during execution of measurement mode M.

FIG. 4 shows an experimental diagram of implementing the method according to the invention, during execution of cleaning mode N.

FIG. 5A shows curves of the odor signals sₙ^{(a)}(ti) measured as a function of time, by all sensors of the electronic nose, in the reference atmosphere formed by the outside air.

FIG. 5B shows curves of the odor signals sₙ^{(c)}(ti) measured as a function of time, by all sensors of the electronic nose, in the atmosphere of the oven containing the product to be cooked.

FIG. 6A shows raw curves sₙ(ti) obtained by finding the difference between the curves of FIG. 5A and those of FIG. 5B: sₙ^{(c)}(ti)−sₙ^{(a)}(ti).

FIG. 6B shows normalized curves obtained by calculating the min-max norm snormₙ(ti)=[sₙ(ti)−minₙ{sₙ(ti)}]/[maxₙ{sₙ(ti)}−minₙ{sₙ(ti)}]; snorm being between 0 and 1.

FIG. 7 shows a control curve giving the odor signals measured as a function of time, by the electronic nose after cooking, without implementation of the method according to the invention.

FIG. 8 shows a curve giving the odor signals measured as a function of time, by the electronic nose after cooking, with implementation of the method according to the invention.

### DETAILED DESCRIPTION

A preferred embodiment of the device according to the invention is described below as a non-limiting example.

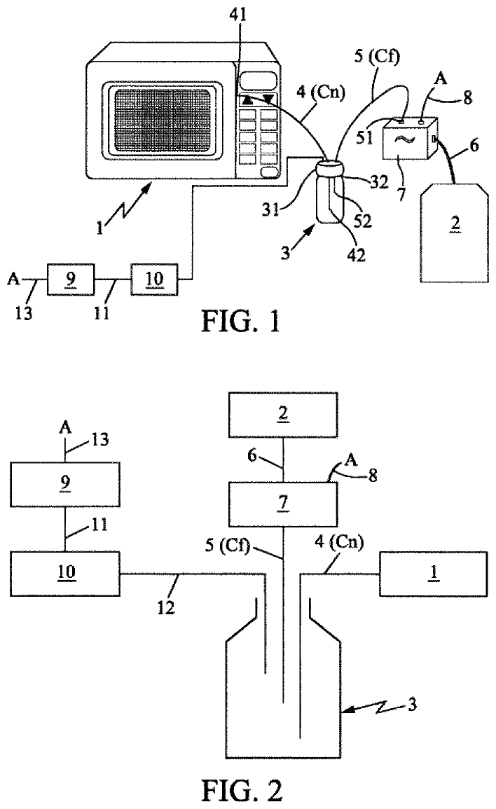

As shown in FIG. 1, the cooking device comprises an oven 1, an electronic nose 2, a removal chamber 3, a communicating pipe Cf 5-6 between the removal chamber 3 and the oven 1, and communicating pipes Cn 4 connecting the removal chamber 3 to the electronic nose 2.

The oven 1 may for example be a commercial electric oven with a power of 1600 W and a capacity of 30 liters. It is intended for cooking a physical body not shown in the figures, which may for example be a food product, such as the Nestlé® brand of American cookie dough, “Toll house—Chocolate Chip Cookie Dough”.

The electronic nose 2 is, for example, a bioelectronic nose manufactured and marketed under the brand NeOse® by the company Aryballe. The NeOse® is described in more detail above. this electronic nose 2 is equipped with a pump making it possible to suction gaseous fluid from the interior atmosphere of the oven 1, in order to bring samples of this fluid into the measurement chamber of the sensor.

The removal chamber 3 is a container, for example a glass vial, closed by a screw-on/screw-off cap 31 on the upper opening 32 of the container 3. The capacity of the container may be 20 ml for example.

The cap 31 of the container 3 is traversed by the communicating pipe Cf 5-6 between the removal chamber 3 and the oven 1 and by the communicating pipe Cn 4 participating in the connection of the removal chamber 3 to the electronic nose 2.

This connection also comprises an automatic valve 7 (fluidic system) and the communicating pipe Cn 4 arranged between the valve 7 and the electronic nose 2.

This automatic valve 7 is a 3-way valve, one of the channels being a bypass connected to a pipe 8 leading to the ambient air, denoted “A” in FIGS. 1 to 4.

Pipe Cf-5-6 is a tube of thermoplastic polymer such as PEEK (polyether ether ketone). Pipe Cf-5-6 has a free end 41 and a free end 42. Free end 41 leads into the chamber of the oven 1, in order to be able to collect samples from the interior atmosphere of this oven 1. Free end 42 is inserted into the removal chamber 3. It is located in the lower half of the removal chamber 3.

Pipe Cn-5 and pipe 8 are advantageously tubes of the same type as pipe Cf-5-6. Pipe Cn-5 has a free end 51 and a free end 52. Free end 51 is connected to a port of the valve 7. Free end 52 is inserted into the removal chamber 3. It is located in the upper half of the removal chamber 3.

In this example, free end 52 is located in the first upper third of the removal chamber 3, while free end 42 of pipe Cf-5-6 is located in the second third of the removal chamber 3 counted from the upper opening of the removal chamber 3.

As for pipe Cn-6, it is a polymer tube of Teflon® (PTFE).

This device also comprises a blowing pump 9, a valve 10 (fluidic system), and communicating pipes 11, 12 allowing the propulsion of a flushing fluid, in this case air, to inside the removal chamber 3. This propulsion is provided by the blowing pump 9 which also has an ambient air intake port 13, denoted “A” in FIGS. 1 to 4. This blowing pump 9, for example of flow rate 1 to 100 L/h, may be a “microblower” such as
an aquarium bubbler of the type marketed by the EHEIM company under the name EHEIM 3701 (aquarium air pump 100 L/h/power draw: 3.5 W);
or a “microblower” called the “Microblower MZB3004T04” and marketed by the muRATA company.

This valve 10 may be a two-way valve (1 open position/1 closed position) or a 3-way valve in which one channel is a bypass leading to the ambient air.

The oven 1, the electronic nose 2, the blowing pump 9, and the valves 7 and 10 are connected to a computer capable of controlling them, and of collecting, storing, and processing the odor measurement data and other parameters such as temperature, pressure, and humidity, acquired during implementation of the method according to the invention.

### EXAMPLES

### Example 1

In this example, the device described above is implemented according to the method for qualitative and/or quantitative measurement of the invention.

As shown in the experimental diagram of FIG. 2, the device comprises a cooking oven 1 (characteristics: electric oven with a power of 1600 W and a capacity of 30 liters), a NeOse®_pro_cooking Version 1 electronic nose marketed by the company Aryballe, a removal chamber 3 composed of 20 mm glass vials, a tube made of PDF 6 connecting the electronic nose 2 to an automatic 3-way valve 7 marketed by the Bürkert company. This valve 7 comprises an air A inlet/outlet port 8 (tube made of PEEK) and a tube 5 made of PEEK inserted into the upper half of the removal chamber 3. The interior chamber of the oven 1 is in communication with the removal chamber 3 via a tube 4 made of PEEK inserted to ⅔ of the depth of this removal chamber 3. Also provided is a blowing pump 9 consisting of a “microblower” consisting of a “Microblower MZB3004T04” marketed by the muRATA company. This blowing pump 9 comprises an air A inlet/outlet port 13 (tube made of PEEK) and a tube 11 made of PEEK which places it in communication with a valve 10 {characteristics Bürkert TwinPower Type solenoid valve 6626—3/2-way with separation membrane DN 2.0 (3 bars) or DN 3.0 (2 bars)] marketed by the Bürkert company}. The valve 10 is connected to one end of a tube 12 made of PEEK, the other end being inserted into the removal chamber 3.

Teflon® gaskets are used for the connectors between the tubes and the elements of the device.

The product to be cooked is a Nestlé® brand of American cookie dough, “Toll house—Chocolate Chip Cookie Dough”. A computer is used to control the process.

The measurement is made at a site n comprising the oven 1 with its cooking chamber and a chamber isolated from this cooking chamber and comprising a reference atmosphere formed by the outside ambient air.

Methodology:
-a- Turn on the NeOse 2 and valve 7, 30 minutes before the first experiment.
-b- Check that the NeOse angle is 120°
-c- Set up the device described above
preheat the oven at 120° C. for 15 min;

M mode:
M.II/ cooking is started by placing the cookie dough in oven 1;
M.III/ the following occur continuously:
-m1- at least one sample of the interior atmosphere of the oven 1 is transferred via the removal chamber to the measurement chamber of the NeOse 2;
-m2- at least one odor measurement of the sample is made;
-m3- the sample is discharged from the measurement chamber;
M.(III)ₓ/ Steps -m1-, -m2-, -m3 are repeated x=15 times with a period of 3 min;
M.III-Ref/ The measurement of the signature of odors of the cooking atmosphere is made, relative to a stable reference measurement formed by the reference chamber isolated from the oven 1 and containing outside air.

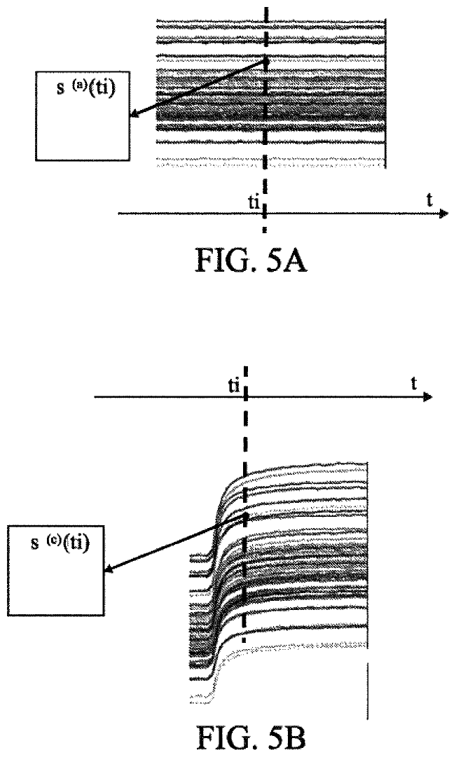

The two sets of time responses shown in the appended FIGS. 5A and 5B are therefore measured simultaneously.

FIG. 5A shows the curves of the signals obtained by the sensors of the electronic nose (n sites) in the reference atmosphere, i.e. the outside air.

FIG. 5B shows the curves of the signals obtained by the same sensors of the electronic nose (n sites), in the cooking atmosphere.

At time ti we therefore have the signal Sₙ^{(c)(ti)} for the reference atmosphere (outside air), and the signal Sₙ^{(c)(ti)} for the cooking atmosphere. The processing of these two sets of time responses in FIGS. 5A & 5B is carried out as follows:

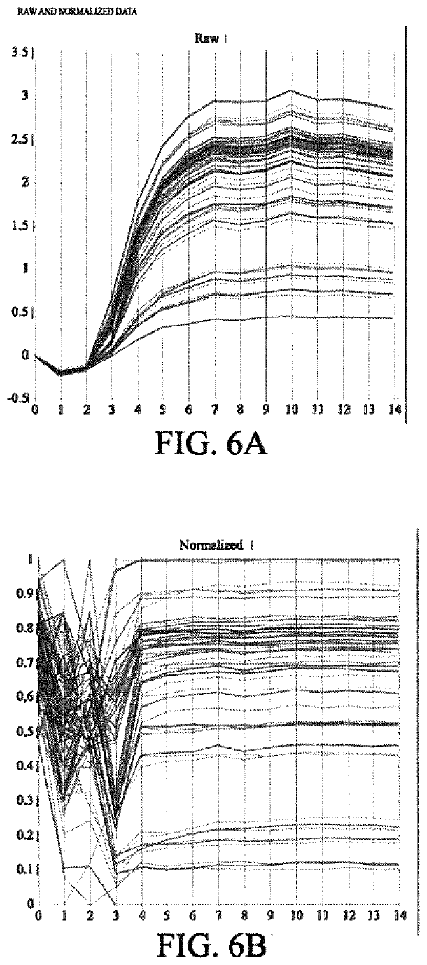

Raw curve (FIG. 6A) by calculating the difference d2: sₙ^{(c)}(ti)−<sₙ^{(a)}(ti)>_{Pm}, where <sₙ^{(a)}(ti)>_{Pm} is the mean value for site n of the measurement in air in the completed period Pm from 1 to 10 s, for example 10 s.

Normalized curve (FIG. 6B) by calculating the min-max norm in order to detect the appearance of scents, i.e. the formation of an odor, with snormₙ(ti)=[sₙ(ti)−minₙ{sₙ(ti)}]/[maxₙ{sₙ(ti)}−minₙ{sₙ(ti)}]; snorm being between 0 and 1.

The curves of FIGS. 6A & 6B show an abrupt difference which corresponds to the appearance of a cooking odor signature.
M.IV/ The cooking is controlled according to a cooking temperature adjustment setpoint, and optionally according to a cooking duration setpoint, taking into account the odor measurements carried out in phase M.III/ and processed as described in phase M.III-Ref/ above.

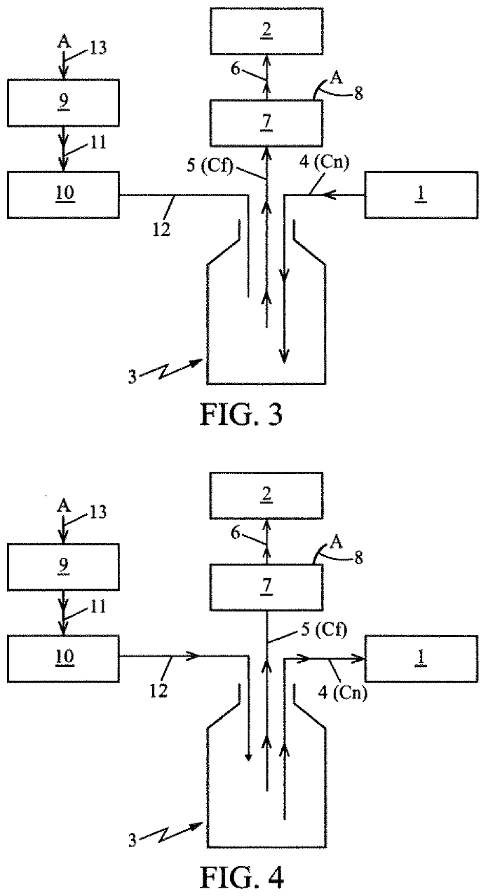

In this measurement mode M, as shown in FIG. 3, the electronic nose 2 sniffs samples of the atmosphere from the chamber of the oven 1 in order to measure the odors of these samples in accordance with the methodology described above. The automatic valve 7 is open to allow the stream of samples to flow from the interior of the oven 1 to the electronic nose 2.

Samples of the reference atmosphere are collected via communicating pipes 6 and 8 and valve 7 (fluidic system). The electronic nose 2 can thus alternately sniff cooking air samples coming from the oven 1 and reference air samples, depending on whether valve 7 has closed or opened the corresponding circuit.

The valve 10 associated with the blowing pump 9 is closed if it is a 2-way valve, or in the case of a 3-way valve it directs the stream from the blowing pump 9 to the 3rd channel connected to the ambient air outlet.

According to a variant, the blowing pump can be stopped.
Mode N:
-N0- flushing the interior of the oven 1 by ventilating it;
-N1- flushing the measurement chamber of the nose 2 with ambient air;
-N2- flushing the removal chamber with a flushing fluid consisting of ambient air;
-N3- transferring a portion of the flushing fluid from the removal chamber into the oven.

In this cleaning mode N, as shown in FIG. 4, the suction pump of the electronic nose 2 sucks in the ambient air. The automatic valve 7 isolates the electronic nose 2 from the rest of the device and places the electronic nose in communication with the ambient air.

The blowing pump 9 propels the flushing stream composed of the ambient atmosphere into the removal chamber 3. The humidity and soiling clogging the walls of the removal chamber 3 and the interior of the communicating pipe 4 are released into the oven and into the ambient air.

In a preferred embodiment, it is possible to replace communicating pipe 4 with a clean pipe or to clean soiled communicating pipe 4, using a cleaning agent consisting for example of a solvent for the soiling which generally consists of fat in the case where the cooked product is a food product. The solvent may for example be ethanol.

### Comparative Example 2

After the cooking in the example below, the oven 1 is switched off, cooled, and ventilated.

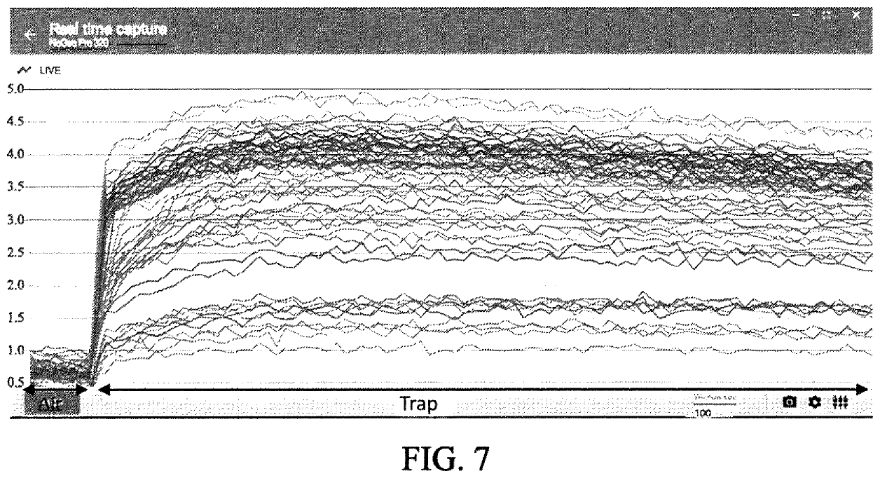

FIG. 7 shows the odor measurement signal obtained with the electronic nose by suctioning from the interior of communicating pipes 6, 5, 4, from the removal chamber 3, and from the chamber of the oven 1, without implementing the cleaning mode N in accordance with the method of the invention.

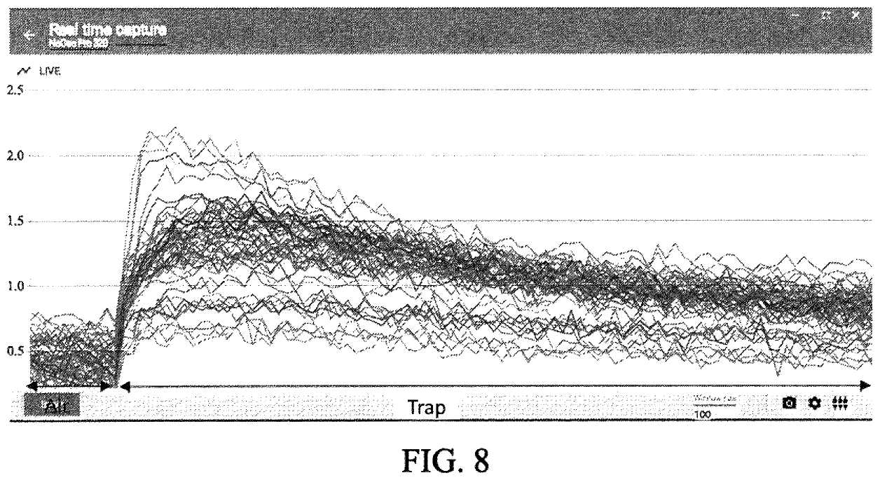

The signal does not decrease significantly. It is more or less stabilized at around 2.2. FIG. 8 shows the odor measurement signal obtained with the electronic nose by suctioning from the interior of communicating pipes 6, 5, 4, from the removal chamber 3, and from the chamber of the oven 1, after implementing the method of the invention, in accordance with the example described above.

The signal then decreases significantly in about thirty seconds.

### LIST OF REFERENCE NUMBERS

-1-: oven; -2-: electronic nose; -3-: removal chamber; -4, 5, 6, 8, 11, 12, 13- communicating pipes; -31-: cap; -32- upper opening of removal chamber 3; -41, 42- pipe ends 4; -51, 52-: pipe ends 5.

1. Method for the qualitative and/or quantitative measurement of odors present in a chamber for transformation under the effect of environmental conditions, preferably cooking or fermentation, of a product, wherein the following are used:
at least one chamber for transformation;
at least one electronic nose provided with at least one measurement chamber comprising at least one sensor, capable of supplying a signal representative of the development of odors during this transformation;
pipes and a fluidic system making it possible to place the measurement chamber in communication with the chamber for transformation, so that samples of the atmosphere of the latter chamber can be transferred into the measurement chamber, where these samples are then brought into contact with the sensor or sensors;
at least one removal chamber connected to the communicating pipes, interposed between the chamber for transformation and the electronic nose and capable of eliminating all or part of any measurement-interfering elements present in the chamber for transformation and therefore also in the samples transferred to the measurement chamber;
said method comprising at least one measurement mode M and at least one cleaning mode N comprising the following steps:
Mode M:
M.I/ optional pre-measurement phase comprising the following steps:
-m1- at least one sample from the atmosphere of the chamber for transformation is transferred into the measurement chamber via the removal chamber;
-m2- at least one odor measurement is carried out on the sample;
-m3- the sample is discharged from the measurement chamber;
steps m1, m2, and m3 preferably taking place continuously;
M.II/ the product is subjected to the conditions to be applied in the chamber for transformation;
M.III/ measurement phase comprising steps m1, m2, and m3;
steps m1, m2, and m3 preferably taking place continuously;
M.(III)ₓ/ optional repetition, with x being an integer >1;
M.IV/ optionally, the transformation is controlled according to an environmental conditions adjustment setpoint for the transformation of the product and/or a transformation stopping setpoint, and optionally according to a transformation duration setpoint, taking into account the odor measurement(s) in M-III;
M.V/ the transformation is stopped;
→Mode N:
-N0- optionally, flushing the chamber for transformation, preferably by ventilating it;
-N1- flushing the measurement chamber with a fluid, preferably ambient air;
-N2- flushing the removal chamber with a fluid, preferably ambient air;
-N3- optionally, transferring at least a portion of the flushing fluid from the removal chamber to the chamber for transformation;
→optionally, an additional mode N is implemented before mode M.

2. Method according to claim 1, wherein the measurement mode M comprises a phase M.III-Ref/ in which:
the measurement of odors from the chamber for transformation is made, at each site n each corresponding to an electronic nose sensor, relative to a reference measurement of a stable reference atmosphere isolated from the transformation atmosphere, this reference atmosphere preferably being the outside air;
two sets of time-response signals are measured simultaneously or sequentially at contiguous periods: a first set comprising the time-response signals Sₙ^{(a)}(ti), Sₙ^{(a)}(ti) corresponding to a time-response signal measured at site n, at time ti, in the reference atmosphere, a second set comprising the time-response signals Sₙ^{(c)}(ti), Sₙ^{(c)}(ti) corresponding to a time-response signal measured at site n, at time ti, in the atmosphere of the chamber;
preferably, at each measurement time, these two sets of signals are processed for each measurement site to obtain:
a raw curve by calculating:
the difference d1: sₙ(ti)=sₙ^{(c)}(ti)=sₙ^{(a)}(ti);
or the difference d2: sₙ^{(c)}(ti)−<sₙ^{(a)}(ti)>_{Pm}, where <sₙ^{(a)}(ti)>_{Pm} is the mean value of the measurement for site n in air in the completed period Pm;
d2 being preferred;
and, optionally, a normalized curve by calculating a norm, preferably the min-max norm, to detect the formation of an odor, with snormₙ(ti)=[sₙ(ti)−minₙ{sₙ(ti)}]/[maxₙ{sₙ(ti)}−minₙ{sₙ(ti)}]; snorm being between 0 and 1.

3. Method according to claim 1, wherein the electronic nose is a bioelectronic nose operating by surface plasmon resonance (SPR) imaging.

4. Method according to claim 1, wherein the cooked product or product to be cooked is a foodstuff which, during transformation, generates measurement-interfering elements selected from the group comprising—ideally consisting of—the following elements: water, fat, carbonaceous substances, and mixtures thereof.

5. Method according to claim 1, wherein the removal chamber is a container into which leads at least one pipe Cf 5-6 in communication with the chamber for transformation and at least one pipe Cn 4 in communication with the electronic nose; the free end of pipe Cf 5-6 preferably being located below the free end of pipe Cn 4 inside the container, knowing that in the case where pipes Cf 5-6 & Cn 4 enter the container through its upper end; the free end of pipe Cf 5-6 even more preferably being located in the lower half of the container, while the free end of pipe Cn 4 even more preferably being located in the upper half of the container.

6. Method according to claim 3, wherein cleaning mode N comprises a step N4 in which:
the pipe Cn in communication with the electronic nose is replaced by a clean pipe Cn,
and/or the inside of this pipe Cn is cleaned using a cleaning fluid.

7. Method according to claim 1, wherein step -m1- is carried out by means of at least one pump, preferably a pump equipping the electronic nose and capable of suctioning the sample coming from the oven.

8. Method according to claim 1, wherein step N2 is carried out by means of at least one blowing pump connected, by at least one communicating pipe, to the interior of the removal chamber and capable of propelling a flushing fluid therein.

9. Method according to claim 7, wherein, during step N2, the flushing fluid is not propelled to the interior of the removal chamber, the pipe connecting the blowing pump to the interior of the chamber being then equipped either with a 2-way valve placed in the closed position, or with a valve comprising 2 channels and at least a 3rd channel bypassing the removal chamber, for the flushing fluid propelled by the blowing pump, said 3rd bypassing channel being placed in the open position.

10. Method according to claim 7, wherein, during step N2, the flushing fluid is propelled to the interior of the removal chamber, the pipe connecting the blowing pump to the interior of the removal chamber then being equipped either with a 2-way valve placed in the open position, or with a valve comprising 2 channels and at least a 3rd channel bypassing the removal chamber, for the flushing fluid propelled by the blowing pump, said 3rd bypassing channel being placed in the closed position.

11. Method according to claim 1, characterized in that it relates to controlling a transformation, preferably a cooking or a fermentation, wherein:
optionally, at least one standard pattern of odors measured during the transformation corresponding to a given transformation of a given product to be transformed, is determined beforehand by calibration;
optionally, this standard pattern is kept in memory;
odor patterns are measured continuously according to mode M during transformation of a same product to be transformed;
these measured patterns are compared with the standard pattern and/or these measured patterns are compared with the previously measured patterns;
the transformation is controlled according to the result of this comparison.

12. Method according to claim 1, wherein the chamber for transformation is a cooking chamber or a fermentation chamber.

13. Method for implementing the method according to claim 1, characterized in that it comprises:
at least one chamber for transformation, preferably cooking or fermentation;
at least one electronic nose provided with at least one measurement chamber comprising at least one sensor, capable of supplying a signal representative of the development of odors during this transformation;
pipes and a fluidic system making it possible to place the measurement chamber in communication with the chamber for transformation, so that samples of the atmosphere of the latter chamber can be transferred into the measurement chamber, where these samples are then brought into contact with the sensor or sensors;
at least one valve on all or part of said pipes;
at least one removal chamber connected to the communicating pipes, interposed between the chamber for transformation and the electronic nose and capable of eliminating all or part of any measurement-interfering elements present in the chamber for transformation and therefore also in the samples transferred to the measurement chamber;
at least one pump, preferably a pump equipping the electronic nose and capable of suctioning samples of the atmosphere of the chamber for transformation;
at least one blowing pump connected, by a communicating pipe, to the interior of the removal chamber and capable of propelling a flushing fluid therein.
